(19) United States
(12) Patent Application Publication
(10) Pub. No.: US 2010/0076295 A1
(43) Pub. Date: Mar. 25, 2010

(54) ELECTRODE SET FOR A PATIENT MONITORING DEVICE

(75) Inventors: James Russel Peterson, Fond du Lac, WI (US); Mark Robert Kohls, New Berlin, WI (US); Robert Edward Henderson, West Bend, WI (US)

Correspondence Address: PETER VOGEL;GE HEALTHCARE, 20225 WATER TOWER BLVD., MAIL STOP W492, BROOKFIELD, WI, 53045, US

(73) Assignee: GENERAL ELECTRIC COMPANY, Schenectady, NY (US)

(21) Appl. No.: 12/236,590

(22) Filed: Sep. 24, 2008

(51) Int. Cl. A61B 5/0408

(52) U.S. Cl. 600/393; 600/509

(57) ABSTRACT

An electrode set is disclosed herein. The electrode set includes a base comprising an insulating material, and a plurality of electrodes disposed on the base. The electrodes comprise a generally exposed conductive material. The electrode set also includes a plurality of conductors disposed on the base. The conductors comprise a generally insulated conductive material, and are each connected to one of the electrodes. The electrodes are positioned on the base relative to each other so that they can be collectively placed on specific portions of a patient's anatomy.

### FIELD OF THE INVENTION

This invention pertains generally to an electrode set for a patient monitoring device such as, for example, an electrocardiograph.

### BACKGROUND OF THE INVENTION

An electrocardiograph is a device adapted to record the electrical activity of a patient's heart over time. The electrocardiograph includes one or more sensors or electrodes adapted for attachment to a patient and configured to sense electrical activity. The electrodes transmit electrical signals pertaining to the cardiac activity via a conductor such as a wire to a controller. The controller may generate a plot referred to as an electrocardiogram (ECG) based on the data from the electrodes.

One problem with conventional electrocardiograph devices is that the process of applying the electrodes is labor intensive. The electrodes generally must be placed with a high degree of precision on specific portions of the patient's anatomy. Therefore it can be both difficult and time consuming to ensure each electrode is properly positioned on the patient. Another problem with conventional electrocardiograph devices is that improperly positioned electrodes can cause a resultant ECG to become imprecise or misleading.

### SUMMARY OF THE INVENTION

The above-mentioned shortcomings, disadvantages and problems are addressed herein which will be understood by reading and understanding the following specification.

In an embodiment, an electrode set includes a base comprising an insulating material, and a plurality of electrodes disposed on the base. The electrodes comprise a generally exposed conductive material. The electrode set also includes a plurality of conductors disposed on the base. The conductors comprise a generally insulated conductive material, and are each connected to one of the electrodes. The electrodes are positioned on the base relative to each other so that they can be collectively placed on specific portions of a patient's anatomy.

In another embodiment, an electrocardiograph system includes a controller configured to monitor cardiac electrical activity, and an electrode set connected to the controller. The electrode set includes a base comprising an insulating material, and a plurality of electrocardiograph electrodes disposed on the base. The electrocardiograph electrodes comprise a generally exposed conductive material. The electrode set also includes a plurality of conductors disposed on the base. The conductors comprise a generally insulated conductive material, and are each connected to one of the electrocardiograph electrodes.

In yet another embodiment, a method includes providing a base composed of a first insulating material, securing a first conductive material to predefined regions of the base in order to define a plurality of electrodes, securing a second conductive material to predefined regions of the base in order to define a plurality of conductors, and securing a second insulating material to the plurality of conductors. The method also includes providing a controller connected to the plurality of conductors. The controller is configured to monitor cardiac electrical activity.

Various other features, objects, and advantages of the invention will be made apparent to those skilled in the art from the accompanying drawings and detailed description thereof.

### BRIEF DESCRIPTION OF THE DRAWINGS

FIG. 1 is a schematic illustration of a diagnostic/monitoring system operatively connected to a patient via a twelve lead system in accordance with an embodiment;

FIG. 2 is a plan view of an electrode set in accordance with an embodiment; and

FIG. 3 is a cross-sectional isometric view of an electrode in accordance with an embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

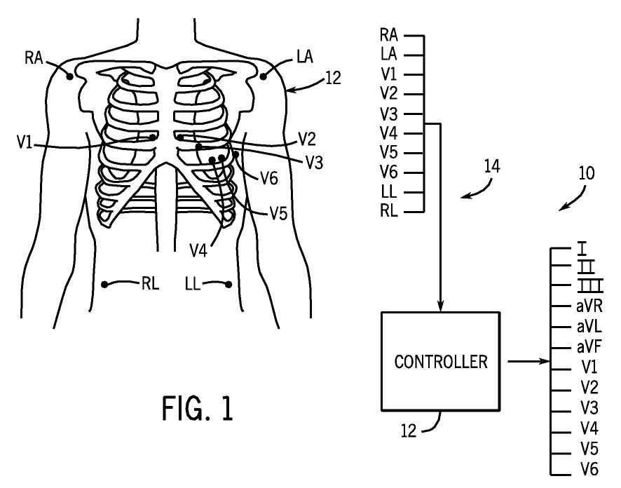

Referring to FIG. 1, a schematically represented diagnostic/monitoring system 10 is shown in accordance with an embodiment. The diagnostic/monitoring system 10 will hereinafter be described as an electrocardiograph 10. It should, however, be appreciated that the diagnostic/monitoring system 10 may comprise other devices such as, for example, an electroencephalograph.

The electrocardiograph 10 includes a controller 12 and an electrode set 14. In the embodiment illustrated in FIG. 1, the electrode set 14 include a right arm electrode RA; a left arm electrode LA; chest electrodes V1, V2, V3, V4, V5 and V6; a right leg electrode RL; and a left electrode leg LL for acquiring a standard twelve lead, ten-electrode electrocardiogram (ECG) signal. The twelve ECG leads include leads I, II, V1, V2, V3, V4, V5 and V6 which are acquired directly from the patient leads, and leads III, aVR, aVL and aVF which are derived using Einthoven's law. In other embodiments, the electrode set 14 may comprise alternative configurations of electrodes and electrode locations to acquire a standard or non-standard ECG signal. For example, an expanded fifteen lead system, including four extra electrodes, can be used to form Frank X, Y and Z leads.

It will be appreciated by those skilled in the art that the electrodes V1, V2, V3, V4, V5 and V6 must be placed with a high degree of precision on the specific portions of the patient's anatomy shown in FIG. 1. Although electrodes RA and LA are shown as being attached to the patient's shoulder, they are more commonly applied at the patient's wrists. Similarly, although the electrodes RL and LL are shown as being attached to the patient's hips, they are more commonly applied at the patient's ankles. If each of the electrodes RA, LA, V1, V2, V3, V4, V5, V6, RL and LL are not precisely placed at the appropriate patient location, the resultant ECG leads I, II, III, V1, V2, V3, V4, V5, V6, aVR, aVL and aVF can become imprecise and/or misleading. Additionally, the placement of the electrodes RA, LA, V1, V2, V3, V4, V5, V6, RL and LL in the manner described requires a great deal of skill and time.

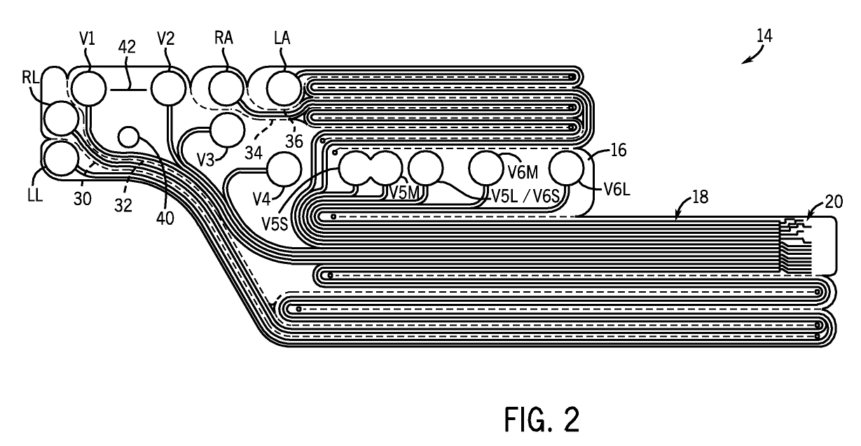

Referring to FIG. 2, the electrode set 14 is shown in more detail. According to one embodiment, the electrode set 14 comprises a die-cut flexible circuit. For purposes of this disclosure, a flexible circuit is defined as a patterned arrangement of printed wiring utilizing a flexible base material. It should be appreciated that die-cut flexible circuits can be inexpensively manufactured such that it may be economically feasible to dispose of the electrode set 14 after each use. Advantageously, a disposable electrode set eliminates or greatly reduces the likelihood of cross-patient infection transmission. For purposes of this disclosure, a disposable electrode set will be defined as one that can be produced for less than five US dollars as measured in the year 2008.

The electrode set 14 includes a base 16, a plurality of sensors or electrodes RA, LA, V1, V2, V3, V4, V5s, V5m, V5l/V6s, V6m, V6l, RL and LL, a plurality of conductors 18, and a connector 20. The electrodes V5s, V5m, V5l/V6s, V6m, and V6l represent an optional electrode configuration wherein each electrode is adapted for use with a patient of a specific size in order to accommodate a variety of different patient sizes. More precisely, in order to maintain the electrode placement depicted in FIG. 1 on a small patient, electrodes V5sand V5l/V6s would be applied to the patient while electrodes V5m, V6m and V6l would remain disconnected. In order to maintain the electrode placement depicted in FIG. 1 on a medium sized patient, electrodes V5m and V6m would be applied to the patient while electrodes V5s, V5l/V6s and V6l would remain disconnected. In order to maintain the electrode placement depicted in FIG. 1 on a large patient, electrodes V5l/V6s and V6l would be applied to the patient while electrodes V5s, V5mand V6m would remain disconnected.

Alternatively, the electrode configuration comprising electrodes V5s, V5m, V5l/V6s, V6m, and V6l may be replaced by a single V5 electrode (not shown) and a single V6 electrode (not shown). As an example, an electrode set 14 adapted to exclusively accommodate medium sized patients may comprise a single V5 electrode disposed at the position currently occupied by electrode V5m, and a single V6 electrode disposed at the position currently occupied by electrode V6m.

The base 16 may comprise a flexible or bendable insulating material such as polyimide, polyester, or polyethylene napthalate. For purposes of this disclosure, an insulating material refers to a dielectric or generally non-conducting material. The flexible nature of the base 16 allows it to more closely conform to the anatomy of a given patient. The electrodes RA, LA, V1, V2, V3, V4, V5s, V5m, V5l/V6s, V6m, V6l, RL and LL, and the connector 20 comprise an exposed conductive material that is printed or etched onto the base 16. As an example, the electrodes RA, LA, V1, V2, V3, V4, V5s, V5m, V5l/V6s, V6m, V6l, RL and LL, and the connector 20 may comprise high elongation copper, roll annealed copper, silver conductive ink, or any of a variety of different metal foils. The conductors 18 may comprise a conductive material printed or etched onto the base 16 that is subsequently covered by an insulating material.

The torso electrodes V1-V6l are printed on the base 16 at a relative position adapted to maintain the electrode placement depicted in FIG. 1 when applied to a patient. Accordingly, a user can position the electrode set 14 as a single unit and thereafter apply all six torso electrodes to the patient based on their relative position on the base 16 rather than individually positioning each of the torso electrodes. In other words, a user can position all six torso electrodes at the same time thereby increasing efficiency and reducing the likelihood of error as compared with known systems requiring the positioning of each electrode individually.

According to one embodiment, the electrode set 14 includes perforated section 30 disposed between electrodes RL and LL; perforated section 32 disposed between electrode RL and the electrodes V1-V6l; perforated section 34 disposed between electrode RA and the electrodes V1-V6l; and perforated section 36 disposed between electrodes RA and LA. The perforated sections 30-36 may be formed during the die-cutting process, and are configured to simplify the process of separating individual electrodes from the remainder of the electrode set 14. As an example, the electrode LL can be conveniently separated from the remainder of the electrode set 14 at the perforated section 30 in order to facilitate the application of the electrode LL at the patient's left ankle while the electrodes V1-V6l are applied to the patient's torso.

According to another embodiment, the electrode set 14 includes an electrode positioning feature 40 and an electrode alignment feature 42. The electrode positioning feature 40 comprises an aperture defined by the base 16, and which may be formed during the die-cutting operation. The electrode positioning feature 40 is located relative to the torso electrodes V1-V6l such that when the patient's sternum is visible through the electrode positioning feature 40, the torso electrodes V1-V6l will be properly positioned relative to the patient. The electrode alignment feature 42 comprises a generally straight line printed on the base 16. The electrode alignment feature 42 is oriented relative to the electrode positioning feature 40 and the torso electrodes V1-V6l such that when the electrode alignment feature 42 is substantially horizontally disposed relative to the patient, the torso electrodes V1-V6l are properly aligned relative to the patient. It should be appreciated that by both positioning and aligning the electrode set 14 in the manner described, the torso electrodes V1-V6l can be placed with a high degree of precision on the specific portions of the patient's anatomy shown in FIG. 1.

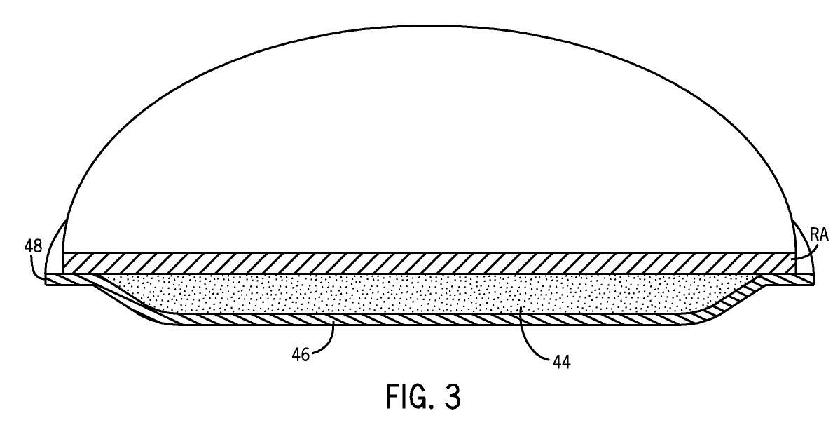

Referring to FIG. 3, a cross-sectional isometric view of the electrode RA is shown in accordance with an optional embodiment. Electrode RA is depicted for illustrative purposes, and it should be appreciated that the remaining electrodes LA, V1, V2, V3, V4, V5s, V5m, V5l/V6s, V6m, V6l, RL and LL may be similarly configured. A conductive gel 44 and a liner 46 are secured to the electrode RA as will be described in detail hereinafter.

The conductive gel 44 provides a conductive medium between the surface of the patient's skin and the electrode RA in order to more accurately monitor a patient's cardiac electrical activity. The conductive gel 44 may also provide adhesion in order to retain the electrode RA after it has been applied. The conductive gel 44 can be applied directly onto the electrode RA as part of the electrode set 14 manufacturing process. The liner 46 is configured to protect the conductive gel 44 until the electrode RA is applied to a patient. The liner 46 is secured to the periphery of the electrode RA with adhesive such that the conductive gel 44 is disposed therebetween. The liner 46 may include a release tab 48 so that a user can conveniently separate the liner 46 from the electrode RA just before the electrode RA is applied to a patient. According to an alternate embodiment, a single large liner (not shown) may be implemented to protect multiple electrodes (e.g., the torso electrodes V1-V6l).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

I claim:

1. An electrode set comprising:
a base comprising an insulating material;
a plurality of electrodes disposed on the base, said plurality of electrodes comprising a generally exposed conductive material; and
a plurality of conductors disposed on the base, said plurality of conductors comprising a generally insulated conductive material, each of said plurality of conductors being connected to one of said plurality of electrodes;
wherein said plurality of electrodes are positioned on the base relative to each other such that they can be collectively placed on specific portions of a patient's anatomy.

2. The electrode set of claim 1, further comprising an electrode positioning feature defined by the base.

3. The electrode set of claim 1, further comprising an electrode alignment feature disposed on the base.

4. The electrode set of claim 1, further comprising a perforated section defined by the base, said perforated section configured to facilitate the separation of one of the plurality of electrodes from the other of the plurality of electrodes such that said one of the plurality of electrodes can be secured to a remotely located portion of a patient's anatomy.

5. The electrode set of claim 1, wherein said plurality of electrodes are printed or etched onto the base.

6. The electrode set of claim 1, further comprising a liner secured to one of the plurality of electrodes.

7. The electrode set of claim 6, further comprising a conductive gel disposed between said liner and said one of the plurality of electrodes.

8. The electrode set of claim 1, wherein the electrode set is disposable.

9. The electrode set of claim 1, wherein said plurality of electrodes comprise one or more electrodes adapted for use exclusively with a specific patient size such that said plurality of electrodes can accommodate multiple patient sizes.

10. An electrocardiograph system comprising:
a controller configured to monitor cardiac electrical activity; and
an electrode set connected to the controller comprising:
a base comprising an insulating material;
a plurality of electrocardiograph electrodes disposed on the base, said plurality of electrocardiograph electrodes comprising a generally exposed conductive material; and
a plurality of conductors disposed on the base, said plurality of conductors comprising a generally insulated conductive material, each of said plurality of conductors being connected to one of said plurality of electrocardiograph electrodes.

11. The electrocardiograph system of claim 10, wherein said plurality of electrocardiograph electrodes are positioned on the base relative to each other such that they can be collectively placed on specific portions of a patient's anatomy.

12. The electrocardiograph system of claim 11, wherein said plurality of electrocardiograph electrodes comprise one or more electrodes adapted for use exclusively with a specific patient size such that said plurality of electrocardiograph electrodes can accommodate multiple patient sizes.

13. The electrocardiograph system of claim 10, wherein the electrode set further comprises an electrode positioning feature defined by the base, and an electrode alignment feature disposed on the base.

14. The electrocardiograph system of claim 10, wherein the electrode set further comprises a perforated section defined by the base, said perforated section configured to facilitate the separation of one of the plurality of electrocardiograph electrodes from the other of the plurality of electrocardiograph electrodes such that said one of the plurality of electrocardiograph electrodes can be secured to a remotely located portion of a patient's anatomy.

15. The electrocardiograph system of claim 10, wherein said plurality of electrocardiograph electrodes are printed or etched onto the base.

16. A method comprising:
providing a base composed of a first insulating material;
securing a first conductive material to predefined regions of the base in order to define a plurality of electrodes;
securing a second conductive material to predefined regions of the base in order to define a plurality of conductors;
securing a second insulating material to the plurality of conductors; and
providing a controller connected to the plurality of conductors, wherein said controller is configured to monitor cardiac electrical activity.

17. The method of claim 16, wherein said securing a first conductive material comprises printing or etching the first conductive material.

18. The method of claim 16, wherein said securing a second conductive material comprises printing or etching the second conductive material.

19. The method of claim 16, further comprising providing a perforated section defined by the base, said perforated section configured to facilitate the separation of one of the plurality of electrodes from the other of the plurality of electrodes such that said one of the plurality of electrodes can be secured to a remotely located portion of a patient's anatomy.

20. The method of claim 16, further comprising providing an electrode positioning feature defined by the base, and providing an electrode alignment feature disposed on the base.
